(19) United States
(12) Patent Application Publication
(10) Pub. No.: US 2024/0423512 A1
(43) Pub. Date: Dec. 26, 2024

(54) System and Methods for Measuring Stimulated Raman Scattering

(71) Applicant: Google LLC, Mountain View, CA (US)

(72) Inventors: Christopher David Workman, Baltimore, MD (US); Vahe Tshitoyan, Fremont, CA (US); Altaf Khetani, Denver, CO (US); Cheongyuen William Tsang, Union City, CA (US); Kelly Elizabeth Dobson, Mountain View, CA (US)

(21) Appl. No.: 18/700,512

(22) PCT Filed: Oct. 11, 2022
(86) PCT No.: PCT/US2022/046251

§ 371 Date: Apr. 11, 2024

Related U.S. Application Data
(60) Provisional application No. 63/254,260, filed on Oct. 11, 2021.

(51) Int. Cl. A61B 5/145; A61B 5/1455; G01N 21/65; G01N 33/66

(52) U.S. Cl. CPC A61B 5/14532; A61B 5/1455; G01N 21/65; G01N 2021/655; G01N 33/66; G01N 2201/0612

(57) ABSTRACT

The present disclosure provides computer-implemented methods, systems, and devices for non-invasively measuring a level of an analyte in a user using Stimulated Raman Scattering. A user computing device includes a Raman pump source that emits pump light toward a skin surface of the user at a pump wavelength. The device includes a Stokes source that emits Stokes light toward the skin surface at a plurality of Stokes wavelengths within a window of Raman measurement wavelengths. The Stokes light comprises a plurality of narrowband emissions at a respective plurality of center wavelengths ranging across the window of Raman measurement wavelengths. The device includes a photodetector that measures light that emanates from the skin surface. The device includes a processor that processes the measured light to provide an estimated analyte level of the user.

### FIELD

The present disclosure relates generally to the non-invasive monitoring of molecules in a body of a user.

### BACKGROUND

A number of human medical conditions exist that may result in a need to measure an amount of particular molecule present in an individual. For example, it is currently estimated that around 463 million adults have diabetes. For many of these individuals, periodic monitoring of the amount of glucose present in their bloodstream is a part of normal life in order to avoid serious medical complications. Traditionally, to measure the chemical makeup internal to a user's body, invasive measuring methods (e.g., such as sample tissue extraction or blood draw) have been used. Example techniques include finger-prick blood glucometry and transdermal continuous glucose monitoring (CGM). These systems are often painful and/or expensive to use. For users that have to repeatedly take such measurements, the pain and expense associated with glucose monitoring can significantly affect their quality of life.

### SUMMARY

Aspects and advantages of embodiments of the present disclosure will be set forth in part in the following description, or may be learned from the description, or may be learned through practice of the embodiments.

One example embodiment includes a device for non-invasively measuring a level of an analyte in a user using Stimulated Raman Scattering. The device can include a Raman pump source that emits pump light toward a skin surface of the user at a pump wavelength. The device can further include a Stokes source that emits Stokes light toward the skin surface at a plurality of Stokes wavelengths within a window of Raman measurement wavelengths, wherein the Stokes light comprises a plurality of narrowband emissions at a plurality of respective center wavelengths within the window of Raman measurement wavelengths. The device can further include a photodetector that measures light that emanates from the skin surface. The device can further include a processor that processes the measured light to provide an estimated analyte level of the analyte in the user.

Another example aspect of the present disclosure is a computer-implemented method for non-invasively measuring a level of an analyte in a user using Stimulated Raman Scattering. The method includes comprising emitting, by a Raman pump source, pump light toward a skin surface of the user at a pump wavelength. The method includes emitting, by a Stokes source, Stokes light toward the skin surface at a plurality of Stokes wavelengths within a window of Raman measurement wavelengths, wherein the Stokes light comprises a plurality of narrow band emissions at a respective plurality of center wavelengths ranging across the window of Raman measurement wavelengths. The method includes measuring, by a photodetector, light that emanates from the skin surface. The method includes processing, by a processor, the measured light to provide an estimated analyte level of the user.

Another example aspect of the present disclosure is an analyte estimation system. The system includes a Raman pump source that emits pump light toward a skin surface of a user at a pump wavelength. The system includes a Stokes source that emits Stokes light toward the skin surface at a plurality of Stokes wavelengths within a window of Raman measurement wavelengths, wherein the Stokes light comprises a plurality of narrow band emissions at a respective plurality of center wavelengths ranging across the window of Raman measurement wavelengths. The system includes a photodetector that measures light that emanates from the skin surface. The system includes a processor that processes the measured light to provide an estimated analyte level of the user.

Other example aspects of the present disclosure are directed to systems, apparatus, computer program products (such as tangible, non-transitory computer-readable media but also such as software which is downloadable over a communications network without necessarily being stored in non-transitory form), user interfaces, memory devices, and electronic devices for measuring stimulated Raman scattering using an embedded computing system.

These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE DRAWINGS

A detailed discussion of embodiments directed to one of ordinary skill in the art are set forth in the specification, which refers to the appended figures, in which:

FIG. 1A illustrates a graph of the wavelength and intensity of light resulting from spontaneous Raman scattering when light is projected into a target material;

FIG. 1B illustrates a graph of the wavelength and intensity of light resulting from stimulated Raman scattering when light is projected into a target material;

FIG. 1C illustrates an example analyte estimation system for measuring the analyte in the arm of a user non-invasively in accordance with example embodiments of the present disclosure;

FIG. 1D illustrates an example analyte estimation system with multiple laser diodes for generating light at a particular wavelength within a Stokes range in accordance with an example embodiment of the present disclosure;

FIG. 1E illustrates an example analyte estimation system with a single tunable Stokes laser for generating light at different wavelengths within a Stokes range as needed in accordance with an example embodiment of the present disclosure;

FIG. 2 illustrates an example computing environment including a user computing device in accordance with example embodiments of the present disclosure;

FIG. 3A illustrates an example of different types of photon scattering by matter in accordance with example embodiments of the present disclosure;

FIG. 3B illustrates an example of energy level changes of a molecule as a result of light scattering in accordance with example embodiments of the present disclosure;

FIG. 4 illustrates a relative amount of Rayleigh scattered light and Raman scattered light for a typical analyte;

FIG. 5 illustrates a graph representing the wavelengths of scattered light in accordance with example embodiments of the present disclosure;

FIG. 6 illustrates spontaneous Raman scattering;

FIG. 7 illustrates stimulated Raman scattering;

FIG. 8A illustrates an example graph representing the amount of light that is Raman scattered with spontaneous Raman scattering;

FIG. 8B illustrates an example graph representing the amount of light that is Raman scattered with stimulated Raman scattering;

FIG. 9 illustrates an example configuration of a system for detecting molecules in the user's skin in accordance with example embodiments of the present disclosure;

FIG. 10 illustrates an example system for detecting the presence of a molecule in the skin of the user in accordance with example embodiments of the present disclosure;

FIGS. 11A and 11B represent example molecule detection systems in accordance with example embodiments of the present disclosure;

FIG. 12 illustrates an example system for non-invasively measuring molecules within a user's body in accordance with example embodiments of the present disclosure;

FIG. 13 illustrates an example system with light sources and photodiodes in accordance with example embodiments of the disclosure;

FIG. 14 illustrates a layout of a plurality of light-producing sources in accordance with example embodiments of the present disclosure;

FIG. 15 illustrates an example analyte detection system in accordance with example embodiments of the present disclosure;

FIG. 16 is a flowchart depicting an example process of detecting molecules within a target in accordance with example embodiments of the present disclosure;

FIG. 17 depicts a block diagram of an example data analysis model according to example embodiments of the present disclosure; and

FIG. 18 is a flowchart depicting an example process of detecting analytes within a target tissue in accordance with example embodiments of the present disclosure.

### DETAILED DESCRIPTION

Reference now will be made in detail to embodiments, one or more examples of which are illustrated in the drawings. Each example is provided by way of explanation of the embodiments, not limitation of the present disclosure. In fact, it will be apparent to those skilled in the art that various modifications and variations can be made to the embodiments without departing from the scope or spirit of the present disclosure. For instance, features illustrated or described as part of one embodiment can be used with another embodiment to yield a still further embodiment. Thus, it is intended that aspects of the present disclosure cover such modifications and variations.

Generally, the present disclosure is directed towards a system for enabling non-invasive continuous monitoring of one or more analytes internal to the body of a user. An example system for monitoring analytes in accordance with example embodiments can estimate the amount of an analyte in a target material (e.g., the tissue of a user) using Raman spectroscopy, by projecting light into a target material and measuring the wavelengths and intensity of the light that is emitted out of the material. More particularly, embodiments of the present disclosure enable Stimulated Raman spectroscopy measurements using one or more narrow band Stokes light sources to provide more accurate and efficient monitoring of analytes.

Raman spectroscopy uses Raman scattering to determine whether an analyte is present in a particular target material (e.g., the skin of a user). Raman scattering is an optical process where excitation light can be projected into a target sample by a light source (e.g., a pump laser). Molecules within the target sample can be excited to a higher energy state by the incoming excitation light. The molecule that has been excited can emit a photon, thus lowering the energy of the molecule to a lower energy level. In some examples, the higher energy state can be a virtual excited state such that the molecule is never actually excited to that state. Instead, both the excitation and the relaxation (when the photon is emitted) occur simultaneously via the virtual state.

The majority of this emitted light will have the same wavelength as the light which excited the molecule to a higher energy level. However, in a small number of cases, the molecules can retain some of the energy imparted by the light as vibrational energy. As a result, the light emitted by the molecule can have a different wavelength than the light that excited the molecule originally. This phenomenon is known as Raman scattering. The wavelength range that this scattered light falls within can be referred to as a Stokes range (or an anti-Stokes range if the molecule gains energy or the emitted photon has less energy).

The specific wavelengths present in photons generated by Raman scattering can be determined by the vibrational modes of the chemical bonds of the molecule that was excited by the excitation light and the wavelength of the incoming light. A system that uses Raman scattering to determine the presence of an analyte can implement either spontaneous Raman scattering or stimulated Raman scattering. It should be noted that other types of Raman scattering can be used to perform the systems and methods described herein.

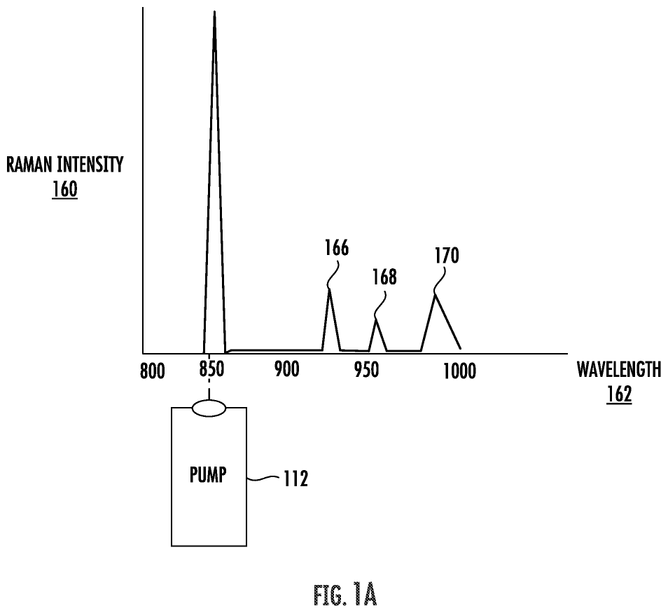

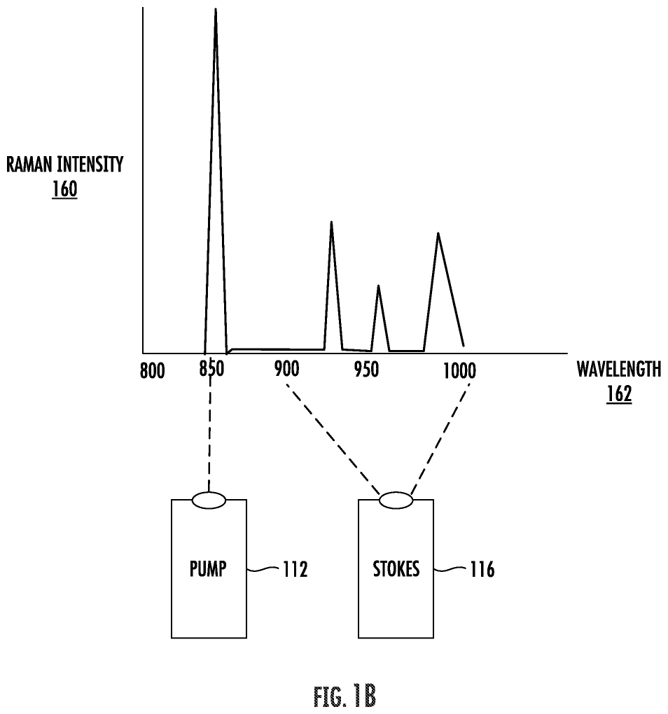

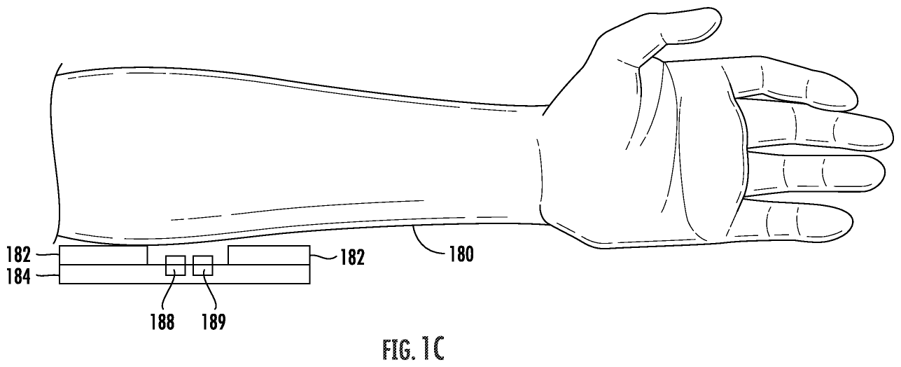

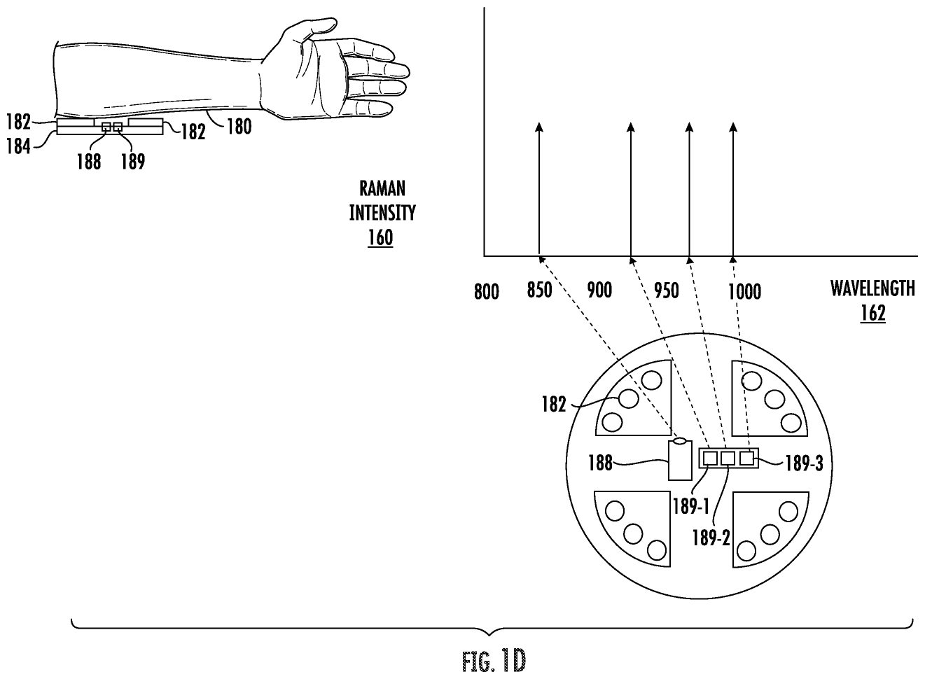

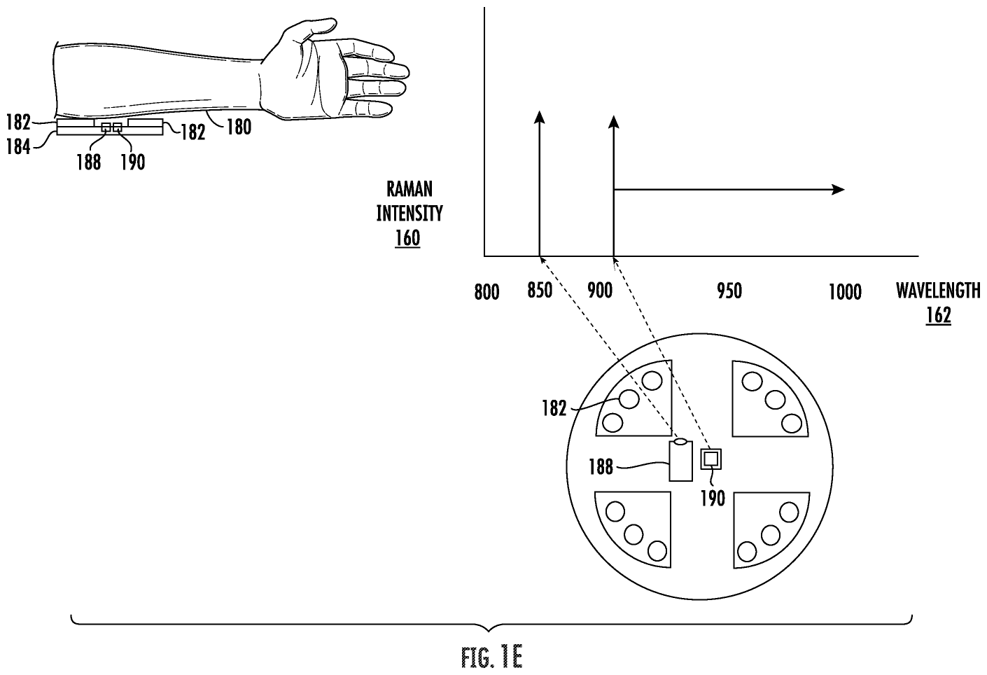

FIG. 1A illustrates a graph of the wavelength 162 and intensity 160 of light resulting from spontaneous Raman scattering when light is projected into a target material. In this example, a pump laser 112 can project light at a particular wavelength (e.g., 850 nanometers). As seen in the example graph, light with high intensity is measured at the wavelength of the pump laser 112. This represents the light that was not Raman scattered and instead has the same wavelength as the light generated by the pump laser 112. The peak that is detected at the particular wavelength (e.g., 850 nanometers) emitted by the Raman pump source can be referred to as a Rayleigh peak. Rayleigh scattering can refer to light that is emitted by the target sample and has the same wavelength as the pump light. Rayleigh scattering is much more common than Raman scattering and thus the intensity of light at the Rayleigh peak can be higher than the intensity measured for light that is the result of Raman scattering.

In this particular example, Raman scattered light is detected between approximately the wavelengths of 900 nanometers and 1000 nanometers. However, other ranges of wavelengths can be measured depending on the specific analyte to be measured. This light is the result of spontaneous Raman scattering in which the light emitted by the target material has a different wavelength than the light produced by the pump laser 112. The range of wavelengths generated by Raman scattering by a particular molecule can be referred to as the Stokes range of that molecule for emitted photons that have a higher wavelength that the originally projected photons or the anti-Stokes range for photons that have a lower wavelength than the originally projected photons. The specific features of the detected Stokes range, including but not limited to the wavelengths at which peak intensity is measured, can be analyzed to generate a Raman signature for the molecule. The Raman signature can represent specific features of a detected Stokes range (e.g., peaks) that are associated with a particular molecule. In this manner, the Raman signature can allow a specific molecule to be identified by analyzing the Stokes range that results when the specific molecule is present in the target material. Not pictured is the anti-Stokes range which can occur on the other side of the Rayleigh peak and represents Raman scattering in which the emitted light has a wavelength lower than the light generated by the pump laser 112. Although much of the present disclosure will be described in terms of the Stokes range, it will be appreciated that the disclosed concepts may be utilized with wavelengths within the anti-Stokes range.

In FIG. 1A, three peaks (166, 168, and 170) are present in the Stokes range. This information, as well as other information about the Stokes range, can be used to determine whether a particular analyte is present in the target material and at what concentrations. However, the amount of Raman scattering that occurs with spontaneous Raman scattering is very low. As a result, the information needed to identify a molecule by its corresponding Stokes range or Raman signature can be difficult to detect.

FIG. 1B illustrates a graph of the wavelength 162 and intensity 160 of light resulting from stimulated Raman scattering when light is projected into a target material. In addition to the pump laser 112, one or more Stokes lasers 116 can project light into a target material. In some examples, the Stokes source(s) 116 can provide light over a wavelength range that is associated with an analyte. Traditionally, the Stokes source can be an LED that provides broadband light at all wavelengths within the wavelength range when the LED is turned on.

As a result of the Stokes laser 116, the Raman response is significantly higher. As a result, the Stokes range, and therefore the Raman signature, of the molecule can more easily be detected and the analyte can more easily be identified.

FIG. 1C illustrates an example analyte estimation system for non-invasively measuring the analyte in the arm of a user in accordance with example embodiments of the present disclosure. A pump laser 188 (e.g., a first light source) can project light into the target material 180. In this example, the target material 180 is the skin and other tissue of the arm of a user. The projected light can be generated by the pump laser 188 at a first wavelength. The analyte estimation system can also include one or more Stokes lasers 189. To facilitate stimulated Raman scattering, the one or more Stokes lasers 189 can be configured to generate light at wavelengths associated with the Raman signature of a target analyte. The pump laser 188 and the one or more Stokes lasers 189 can be vertical-cavity surface-emitting lasers (VCSELs) and can be integrated into a printed circuit board (PCB) 184.

The one or more Stokes lasers 189 can be narrow-band light sources. The pump laser 188 also can be a narrow-band light source in some examples. As used herein, a narrow band light source can project light such that the photons have wavelengths that fall within a narrow range of wavelengths relative to the relevant wavelength range (e.g., Stokes range) associated with the Raman-scattered radiation for the analyte of interest. In some examples, a narrow band light source can be defined based on the percentage of that wavelength range that is generated at a particular point in time. For example, a narrow band laser can generate light that falls within a bandwidth that is 10% of the total wavelength range that the laser can project. For example, if a particular Stokes laser 189 is tunable such that it can generate light within the range of 500 nanometers to 1500 nanometers, a narrow band laser can project light such the wavelength of each photon falls within a 100-nanometer range (e.g., 10% of the total range of the laser). In another example, a narrow band laser can be defined as a laser that projects light with a wavelength bandwidth of 1% of the total wavelength range of the laser. With this definition, the narrow band laser can produce light that falls within a 10-nanometer range (e.g., such that all the projected light has a wavelength within five nanometers of the target wavelength).

In some examples, the Stokes laser 189 can be a single tunable laser that can sweep a narrow band laser over the range of wavelengths that the tunable laser can produce. In some examples, the tunable laser can sweep over the wavelengths in the Stokes range of the target analyte. In another example, the Stokes laser 189 can include a plurality of laser diodes, each of which generates a narrowband of light centered around a wavelength in the Stokes range of the target analyte.

In some examples, the narrow band VCSEL light sources can be tunable over a relatively small range (e.g., approximately 5 nm range) using temperature and electrical current. In this way, the narrow band VCSEL light sources can be enabled to obtain a larger range without the extra components that are typically included in a tunable laser such as additional mechanical components integrated in the chip (e.g. MEMS mirrors) to allow for the wider tunability.

In some examples, other configurations can be used. For example, the analyte estimation system can include more than one pump laser combined with a tunable Stokes laser. Thus, the analyte estimation system can include. 2 pump lasers 188 that are 20 nm apart, and a single tunable Stokes laser 189 that can be tuned over a 20 nm range, resulting in a total of 40 nm range of possible Raman shifts. Additionally, or alternatively, with 3 pump lasers 20 nm apart the range can be tripled. Additionally, or alternatively, one or more fixed wavelength Stokes lasers 189 and a tunable pump laser 188.

The light projected by the pump laser 188 can excite one or more molecules within the target material 180 (e.g., electrons within the molecules are raised to a higher energy level). The molecules can return to a lower energy level and emit one or more photons. A photodetector 182 can detect light emitted from the target material 180. The detected light can be represented in a graph of the intensity of the light at various wavelengths.

The detected light can form a Stokes range for the molecule that was excited by incident light. The spectral intensity at wavelengths across the Stokes range for a particular analyte can form a specific Raman signature. In some examples, a Raman signature can be associated with a specific pattern based on the wavelengths at which peaks are detected, the spacing between peaks, and/or the intensity of light that is detected at one or more wavelengths. The features detected in the Stokes range can be compared to predetermined Raman signatures to determine one or more analytes present in the target material.

Data representing the Stokes range can include information representing the amount of light or intensity of light at each wavelength in a range of wavelengths included in the Stokes range. The data representing the Stokes range can be analyzed to determine one or more features, including peak wavelengths (e.g., wavelengths at which the measured intensity is higher than other nearby wavelengths), troughs, the distances between peaks, the distances between each peak wavelength, and the wavelength of the pump laser, and so on.

FIG. 1D illustrates an example analyte estimation system with multiple laser diodes generating light at one or more particular wavelengths within a Stokes range in accordance with an example embodiment of the present disclosure. As with FIG. 1C, a pump laser 188 can project light into a target material. A plurality of Stokes lasers (189-1, 189-2, and 189-3) can project narrow band light into the target material 180 with a center wavelength different from the center wavelength of the pump laser 188. Photodetectors 182 can be placed such that they measure light being emitted by analytes in the target material 180.

The Stokes lasers 189 can each generate narrow-band light (e.g., the light generated by the Stokes lasers falls within a particular wavelength band which can be represented as a percentage of the total wavelength range of the Stokes laser or as a specific wavelength number such as within 1 nanometer of the target wavelength) for a different wavelength within the Stokes range. In one example embodiment, the pump laser (e.g., the first light source) and the Stokes lasers (e.g., one or more second light sources) are VCSELs that each use 40 milliwatts of power. In other examples, different lasers that use different amounts of power can be used.

It should be noted that light can be measured based on its wavenumber rather than its wavelength. Wavenumber can represent the spatial frequency of an electromagnetic wave and can be measured relative to a base value (e.g., in this case, the light produced by the pump laser can be considered to have a wavenumber of 0 and the light produced by the one or more second sources can be given a wavenumber relative to the wavenumber of the light produced by the pump laser).

FIG. 1E illustrates an example analyte estimation system with a single tunable Stokes laser 189 generating light at different wavelengths within a Stokes range in accordance with an example embodiment of the present disclosure. As with FIG. 1C, a pump laser 188 can project light into a target material. A tunable Stokes laser 190 can generate light that can project a narrowband of light into the target material 180 that can be adjusted to any wavelength within a Stokes range of interest. In some examples, the tunable laser can start with a narrow band of light at the low end of the Stokes range and adjust the wavelengths such that they sweep along the entire Stokes range without widening the narrow band of light wavelengths at any point. Photodetectors 182 can be placed such that they measure light being emitted by analytes in the target material 180.

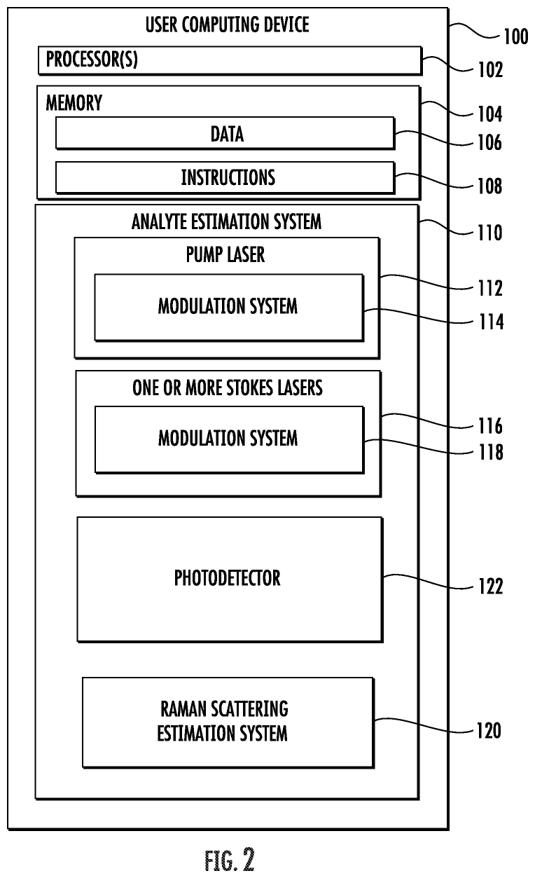

FIG. 2 illustrates an example computing environment including a computing device 100 in accordance with example embodiments of the present disclosure. The computing device 100 can include an analyte estimation system for non-invasively determining the presence and amount of one or more analytes internal to a user. In some examples, the computing device 100 can be a user computing device such as a smartphone or a wearable computing device. In other examples, the computing device 100 can be a computing device intended for home use and not for portability. In this example, the user computing device 100 can include one or more processors 102, memory 104, and an analyte estimation system 110.

In more detail, the one or more processors 102 can be any suitable processing device for a computing device 100. For example, such a processor can include one or more of: one or more processor cores, a microprocessor, an ASIC, an FPGA, a controller, a microcontroller, etc. The one or more processors can be one processor or a plurality of processors that are operatively connected. The memory 104 can include one or more non-transitory computer-readable storage media, such as RAM, ROM, EEPROM, EPROM, flash memory devices, etc., and combinations thereof.

In particular, in some devices, memory 104 can store instructions 108 for implementing the analyte estimation system 110. It will be appreciated that the term “system” can refer to specialized hardware, computer logic that executes on a more general processor, or some combination thereof. Thus, a system can be implemented in hardware, application-specific circuits, firmware, and/or software controlling a general-purpose processor. In one embodiment, the system can be implemented as program code files stored on the storage device, loaded into memory, and executed by a processor or can be provided from computer program products, for example, computer-executable instructions, that are stored in a tangible computer-readable storage medium such as RAM, hard disk or optical or magnetic media.

Memory 104 can also include data 106 that can be retrieved, manipulated, created, or stored by the one or more processor(s) 102. In some example embodiments, such data can be accessed and used as input to the analyte estimation system 110. In some examples, the memory 104 can include data used to perform one or more processes and instructions that describe how those processes can be performed.

In some examples, the analyte estimation system 110 can include a pump laser 112, one or more Stokes lasers 116, a photodetector 122, and a Raman scattering estimation system 120. Although not pictured, the analyte estimation system 110 can also include an optical filter and one or more optical lenses (e.g., micro lenses) to focus the lasers on the same area (e.g., the same portion of the user's skin). The pump laser 112 (e.g., a first light source) can be a laser diode that emits light (e.g., a stream of photons) within a narrow wavelength band such that the emitted light has a particular wavelength within a certain range of the target wavelength. In some examples, the pump laser can produce narrow band light with an average wavelength of 780 nanometers. Other wavelengths of a pump laser 112 may be used, with the wavelengths of the one or more Stokes lasers 116 being determined based, at least in part, on the wavelength of the pump laser 112. In some examples, the pump laser 112 can be a vertical-cavity surface-emitting laser (VCSEL) included in a semiconductor chip. In some examples, the wavelength of the light emitted by the pump laser 112 is 850 nanometers. Other wavelengths can be used.

The pump laser 112 can include (or be associated with) a modulation system 114. The modulation system 114 can include a waveform generator that can produce a waveform that can be used to modulate the light produced by the pump laser 112. The pump laser can be referred to as a first light source. By modulating the light produced by the pump laser 112, the analyte estimation system 110 can differentiate (e.g., using a filter or lock-in amplifier) between light that the target material emits after being excited by the light that originated from the pump laser 112 and the light that the target material emits after being excited by light that originates from the one or more Stokes lasers 116.

The one or more Stokes lasers 116 can include a tunable laser that can produce light with a wavelength within a predetermined range as needed. Thus, the tunable laser can be adjusted such that the wavelength of the light produced by the light source can change within a range. For example, in some examples, the tunable laser can be adjusted to emit light with a wavelength that can vary from 910 nanometers to 980 nanometers. In some examples, the wavelength of the light produced by the tunable laser can be determined based on the Raman signature of a particular analyte that the analyte estimation system 110 is trying to identify. In some examples, both the pump laser and the one or more Stokes lasers can use about 40 milliwatts of power to operate.

In some examples, the one or more Stokes lasers can include a modulation system 114. Thus, in some configurations, the pump laser 112 is modulated to distinguish the light produced by the pump laser 112 from the light produced by the one or more Stokes lasers 116. In other examples, the one or more Stokes lasers 116 are modulated to distinguish between the two light sources.

In some examples, the one or more Stokes lasers 116 can provide light with a wavelength tuned to the Raman signature of a particular analyte that the analyte estimation system 110 is trying to identify (e.g., glucose). By providing additional light (e.g., a stream of photons) with a wavelength determined based on the Raman signature of the analyte, the analyte estimation system 110 can enable stimulated Raman scattering to occur. Stimulated Raman scattering can result in the light provided by the one or more Stokes lasers 116 stimulating more Raman scattering than would be expected without the additional light provided by the one more Stokes lasers 116. Thus, introducing the light provided by the one or more Stokes lasers 116 can increase the detectability of a particular analyte in the sample material because the probability of Raman scattering is increased.

In some examples, the analyte estimation system 110 can include a photodetector 122. The photodetector 122 can be a sensor (e.g., a semiconductor device that converts light (e.g., photons) into electrical current) such as a photodiode. The photodiode can be configured to detect light over a range of wavelengths. In some example embodiments, light can be optically filtered such that only light within a specific wavelength range is detected by the photodetector. An amount of light can also be understood to be the number of photons detected and/or the intensity of the light measured at a particular wavelength.

In some examples, a filter can be employed to remove target-emitted light that is associated with the one or more Stokes lasers 116 such that only light originating from the pump laser 112 is detected. Similarly, an optical filter can filter out light with a wavelength associated with the pump laser 112 such that only target-emitted light that results from the Stokes lasers 116 or Raman scattering is detected by the photodetector. In some examples, a filter (or a lock-in amplifier) can remove modulated light, if the one or more Stokes lasers 116 were modulated, or unmodulated light, if the pump laser 112 was modulated.

The Raman scattering estimation system 120 can be used to detect the amount of light (e.g., the intensity of the light or the number of photons) generated by Raman scattering associated with an analyte in the sample material. In some examples, the Raman scattering estimation system 120 can determine the amount of light (e.g., either the number of photons or the intensity of the light) that has been Raman scattered to identify an analyte in the target material. In a first example, the user computing device can determine the amount of light at the pump wavelength (e.g., a first wavelength) that is lost (stimulated Raman loss). Alternatively, the user computing device can determine the amount of light at the Stokes associated wavelength that is gained (e.g., stimulated Raman gain). Either measurement or their combination can be used to estimate the amount of a particular analyte in the target material (e.g., a user's skin). A detected Stokes range can be compared to a reference spectrum to noninvasively measure the presence or absence of a target analyte.

For example, the sample material can be a portion of a user's body. The analyte can be, for example, glucose. Based on the amount of light having the predetermined second wavelength, the Raman scattering estimation system 120 can estimate the amount of the analyte in the target sample. In some examples, the estimated amount of the analyte can be presented for display to a user.

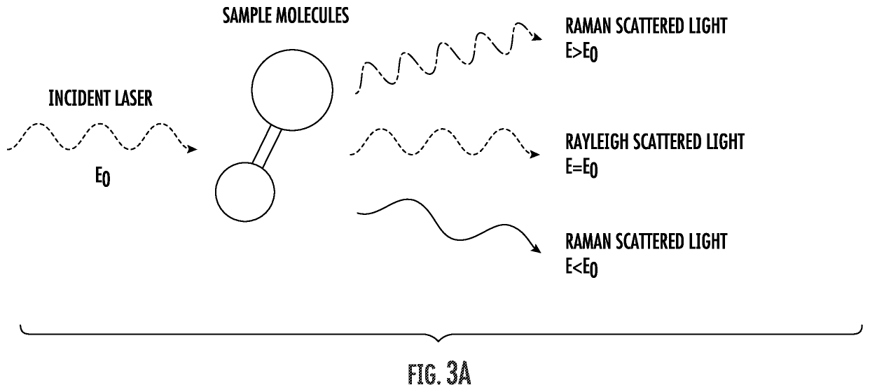

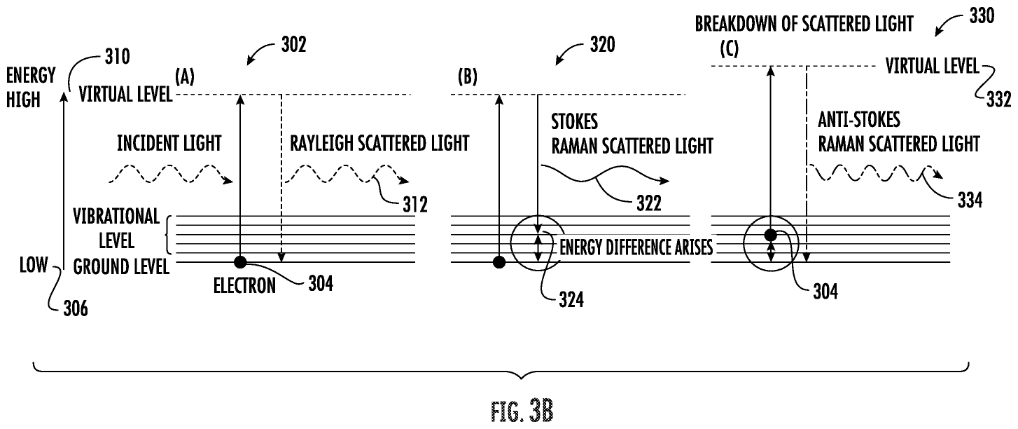

FIG. 3A illustrates an example of different types of photon scattering by matter. As seen in this example, an incoming photon 200 (e.g., the incident light) can interact with a particular sample molecule. One or more photons of the incident laser can interact with the sample molecules (e.g., with electrons in the sample molecules), temporarily raising the electron's energy level. When the energy level of the electron returns to its lower level, a photon is emitted. In some examples, this process can be referred to as scattering. Scattering can include Raleigh scattering and Raman scattering. In Raleigh scattering (which is a type of elastic scattering), the emitted photons have the same energy (and thus the same wavelength) as the incident photon but with the trajectory of the photon potentially altered. In Raman scattering, the energy level of the electron is changed, such that when the photon is emitted, the energy level (and thus the wavelength) of the photon is different than the incident photon. As a result, the presence of a particular sample molecule can be determined based on the presence or absence of Raman scattered light with particular altered wavelengths.

In some examples, the energy level of the target molecule can increase, resulting in a decrease in the energy of the Raman scattered light (referred to as Stokes Raman scattered light), or the energy level of the target and molecule can decrease, resulting in an increase in the energy of the Raman scattered light (referred to as anti-Stokes Raman scattered light).

It should be noted that, if the incident light has a consistent wavelength, the Raman scattered light will result in a particular Stokes range. The Stokes range can have a consistent Raman signature which can enable an analyte estimation system to distinguish the Raman scattering that results from one analyte from the Raman scattering that results from a second analyte. Thus, identifying the presence of a particular Raman signature can be used to estimate the presence of the target analyte and may also be used to estimate the amount of that analyte in the target substance.

FIG. 3B illustrates an example of energy level changes of an analyte as a result of light scattering. As seen herein, an electron 304 can have a first energy level 306 (e.g., a base energy level). In response to interactions with the incident light, the energy level of the electron increases. After a period of time, the electron 304 can emit a photon and return to a lower first energy level 306.

In a first example 302, the electron 304 is initially at a first energy level 306 (a low energy level). In response to the incident light, the electron 304 gains energy to a second energy level 310 higher than the first energy level 306. The electron 304 can emit the light as Rayleigh scattered light 312. In this example 302, the energy of the Rayleigh scattered light 312 is the same as the incident light (and thus has the same wavelength). The electron 304 can return to the first energy level 306 such that the total energy level of the system is maintained.

In a second example 320, the electron 304 is initially at a first energy level 306 (a low energy level). In response to the incident light, the electron 304 gains energy to a second energy level 310 higher than the first energy level 306. However, in this example, some of the energy is gained by the molecule as vibrational energy. As a result, when the Raman scattered light is emitted by the electron 304 the energy (and thus wavelength) of the emitted photon 322 is reduced but does not fall back down to the first energy level 306. Instead, the electron remains at the third energy level 324 which is higher than the first energy level 306 but lower than the second energy level 310. Thus the total energy of the system is maintained because the electron 304 ends up at a third energy level 324 which is higher than the first energy level 306 but the scattered photon 322 is at a lower energy level than the incident light.

In a third example 330, the electron 304 is initially at a fourth energy level 332 higher than the first energy level 306. In response to the incident light, the electron 304 gains energy to a fifth energy level 334 higher than the second energy level 310. When the Raman scattered light is emitted, the electron 304 returns to the first energy level 306 having lost energy from its initial starting position at the fourth energy level 332. The scattered light 334 (referred to as anti-Stokes Raman scattered light) can have a higher energy level than the incident light.

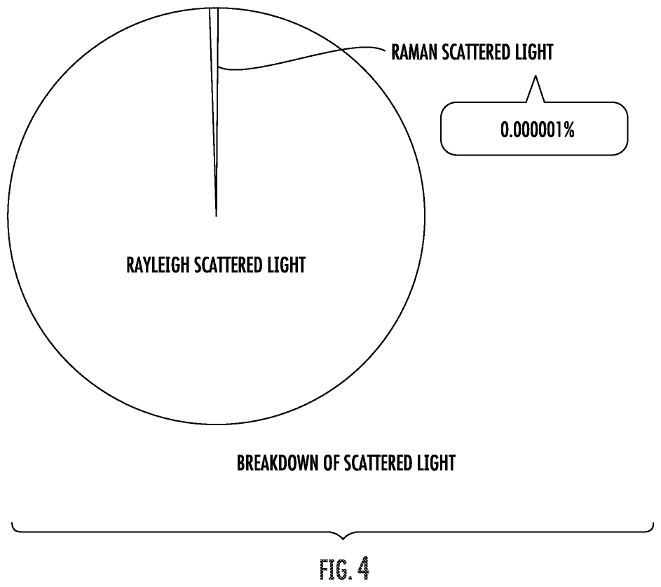

FIG. 4 illustrates a relative amount of scattered light. In this example, it is clear that the amount of Raman scattered light is a tiny fraction of the total amount of scattered light. Thus if Raleigh scattered light represents more than 99.99 percent of all the scattered light, Raman scattered light can represent as little as 0.000001 percent of the scattered light. As such, the amount of Raman scattered light is much less than the total amount of scattered light. As a result, any technique to increase the amount of Raman scattered light can result in significant improvements in the ability of the detection system to determine whether or not an analyte is present.

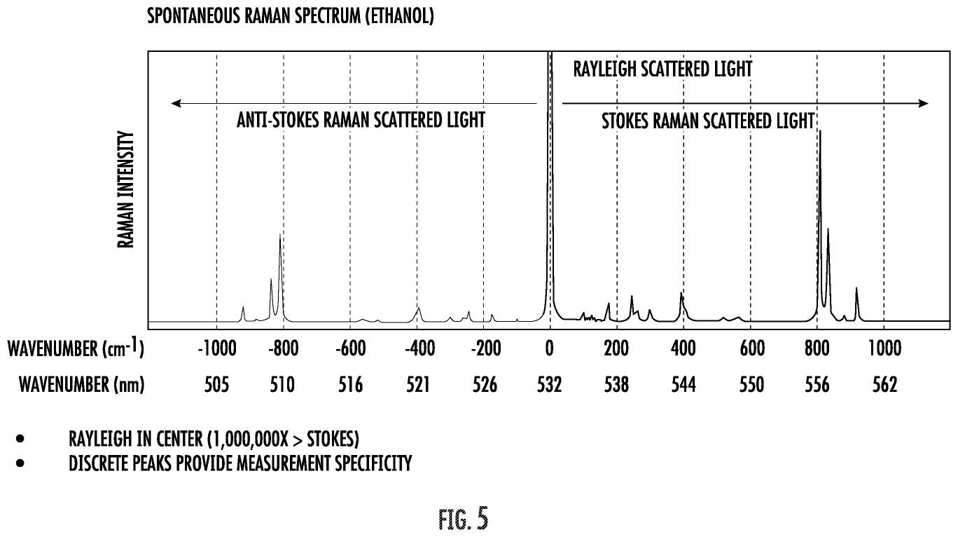

FIG. 5 illustrates a graph representing the wavelengths of scattered light in a Stokes range. In this example graph, the detected scattered light can be represented as a Stokes range that shows the wavelength of the detected light and its intensity. As can be seen, the majority of scattered light can be Rayleigh scattered light with the wavelength the same as the wavelength of the incident light generated by the pump laser. Thus, the peak at the 532-nanometer wavelength (wavenumber 0) is very high. Other peaks (e.g., at 800 nanometers and so on) can represent Stokes Raman scattered light (in which the light has lost energy) or anti-Stokes Raman scattered light (in which the light has gained energy) in a Stokes range associated with an analyte.

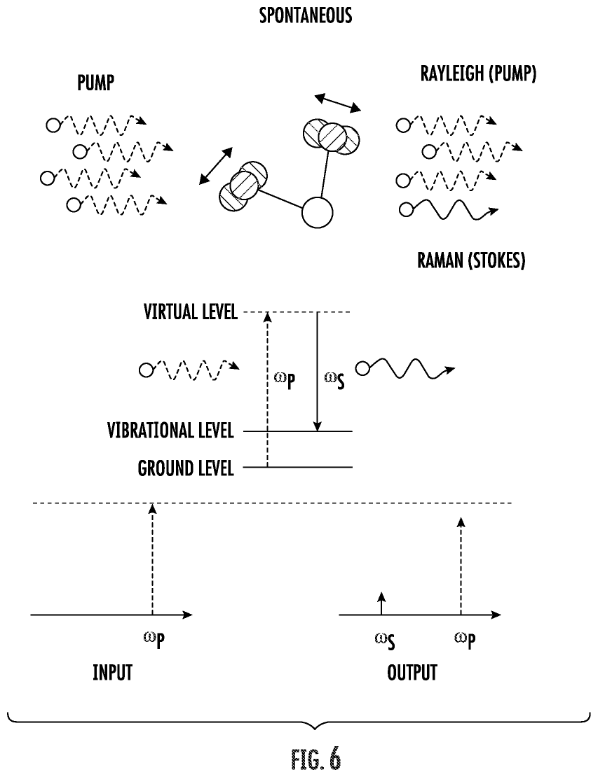

FIG. 6 illustrates spontaneous Raman scattering. For spontaneous Raman scattering, light generated by the first light source (or pump laser) can interact with a particular molecule. A small fraction of the photons that are emitted by the molecule(s) will have less energy and thus have a different wavelength than the incoming photons. As noted above, a particle in the molecule (e.g., an electron) can gain energy increasing from a ground level to a virtual level. In some cases, rather than returning to the ground level, one or more particles in the molecule can retain some energy as vibrational energy. Thus, the photon emitted when the energy lowers has less energy than the incoming photon.

As a result, most of the emitted light retains the same wavelength as the incoming light. However, a small fraction of the incoming light is scattered such that the energy level of the scattered light and its wavelengths are different from the incoming light that excited the molecule. As noted above, the Raman signature of the scattered light can be used to determine what analytes are present in the target material.

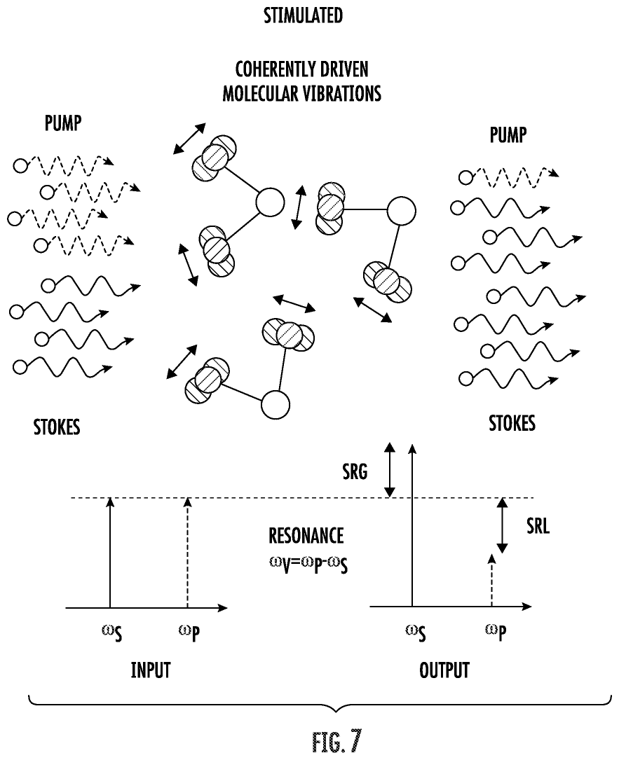

FIG. 7 illustrates stimulated Raman scattering. In the case of stimulated Raman scattering, two or more light sources can be used to amplify or increase the amount of light that is scattered in by Raman scattering, thus increasing the average intensity of the Stokes range. In some examples, a pump laser can generate light with a first wavelength (as with spontaneous Raman scattering). In addition, one or more Stokes lasers can generate light with a second wavelength. The second wavelength can be a wavelength associated with the Raman signature of a particular analyte.

The light with the first wavelength and the light with the second wavelength can be combined and projected towards a target sample. A dichroic mirror (or one or more optical lenses) can be used but is not required. The light with a second wavelength can cause coherently driven molecular vibrations that have the effect of increasing the amount of Raman scattered light. As a result, less light is needed from the pump laser to result in a detectable amount of Raman scattered light.

In some examples, if the generated light can be equally split between the first wavelength and the second wavelength, the light emitted by molecules in the target sample can have more light that has the second wavelength. The difference between the amount of light with the second wavelength that is generated by the one or more Stokes lasers and the amount of light with the second wavelength that is measured after emission can be the amount of light that has been emitted with a different wavelength due to stimulated Raman scattering.

The light generated by the pump laser (or first light source) can excite a particular molecule to a higher energy level. A small fraction of the photons that are emitted by the molecule will be emitted with less energy (e.g., a particle in the molecule can retain some energy as vibrational energy) and with a different wavelength. As noted above, a particle (e.g., an electron) in the molecule can gain energy increasing from a base level of energy to a higher virtual energy level. In some cases, rather than returning to the base level, the one or more particles in the molecule can retain some vibrational energy and thus the photon emitted when the energy level of the electron returns to a lower level has less energy than the incoming photon.

As a result, most of the emitted light retains the same wavelength as the incoming light. However, a small fraction of the incoming light is Raman scattered such that the emitted light has a different energy level and thus a different wavelength. As noted above, the wavelength detected for the emitted light can be used to determine what analytes are present in the target material.

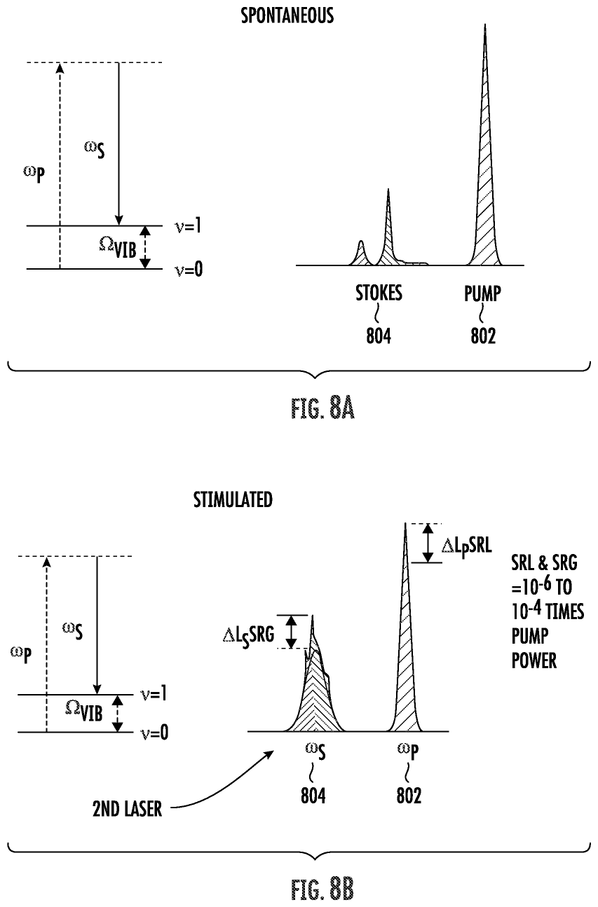

FIG. 8A illustrates an example graph representing the amount of light that is Raman scattered with spontaneous Raman scattering. As noted above, during spontaneous Raman scattering, a single light source can provide light with a first wavelength 802. A small portion of the light provided at the first wavelength 802 can excite a molecule to a higher energy level. Light with a second wavelength 804 can be emitted because some of the energy is retained by particles within one or more target molecules as vibrational energy.

FIG. 8B illustrates an example graph representing the amount of light that is Raman scattered with stimulated Raman scattering. In this example, a pump laser generates light at a first wavelength 802 and a Stokes laser can generate light whose wavelength is the second wavelength 804. As a result of resonance, the amount of Raman scattered light is increased by a first amount 806, making detection of the scattered light more efficient and requiring less power.

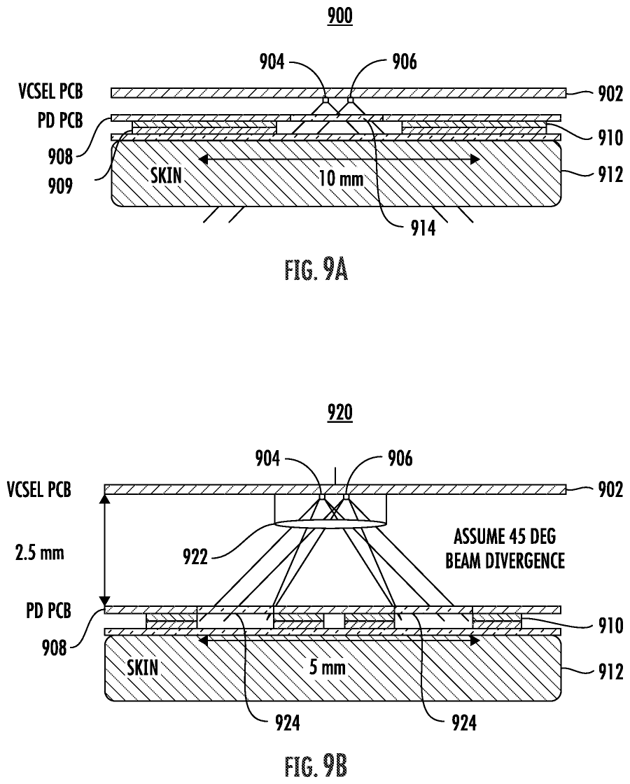

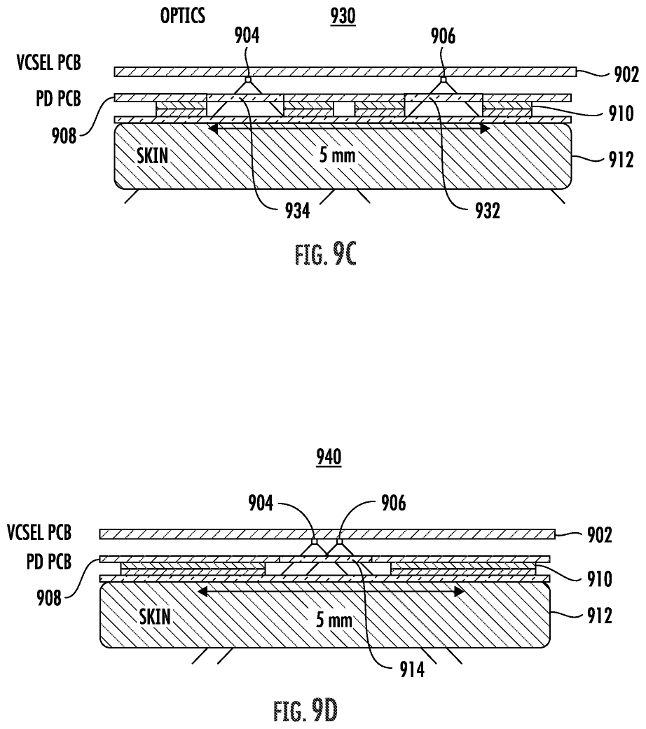

FIG. 9A illustrates an example configuration of a system 900 for detecting analytes in a user's skin in accordance with example embodiments of the present disclosure. In this example, the relevant portion of an analyte estimation system 110 includes two printed circuit boards (PCBs). A first PCB 902 can include two light sources (VCSELs). The first light source 904 can be a pump laser that produces light at one or more first wavelengths and the second light source 906 can be a Stokes laser that produces light at one or more second wavelengths. The second wavelength(s) can be associated with the Raman signature associated with a target analyte.

The second printed circuit board 908 can include one or more photodiodes 910 that are configured to detect light. The one or more photodiodes 910 can produce a signal based on the detected light. In some examples, the signal produced by the photodiode(s) can be processed to remove a modulated portion of the signal generated in response to light produced by the Stokes laser 906. In this manner, the processed signal can represent light produced by Raman scattering of the pump laser 904. The analyte estimation system 110 can be pressed against the skin 912 of a user such that the light is projected down into the skin of a user and molecules included in the skin and/or tissue of the user can emit light into the photodiode 910. It should be noted that two PCBs are used in this example, but in some example embodiments, only a single PCB is used. If so, the photodiodes can be slightly raised from the surface.

The analyte estimation system 110 includes a single window 914 through which the light from both sources passes to interact with the skin 912 of a user. The light interacts with molecules in the user and in response light is emitted from the skin 912 of the user. The emitted light can pass through an optical filter 909 to the photodiode 910.

FIG. 9B illustrates an example configuration of a system 920 for detecting analytes in the user's skin in accordance with example embodiments of the present disclosure. In this example, the relevant portion of the computing system includes two printed circuit boards (PCBs). A first PCB 902 can include two light sources (VCSELs). A first light source 904 can include a pump laser that can produce light at one or more first wavelengths and a second light source 906 can include a Stokes laser that can produce light at one or more second wavelengths. The second wavelength(s) can be associated with the Raman signature of a target analyte. The light from both light sources passes through an optical element 922 (e.g., a lens) before passing through two or more windows 924.

The second printed circuit board 908 can include one or more photodiodes 910 that are configured to detect light. One or more optical filters can be used to filter the received light for one or more target frequencies. For example, a long pass optical filter can be used to pass Stokes frequencies and reject pump frequencies. The one or more photodiodes 910 can produce a signal based on the detected light. In some examples, the signal produced by the photodiode(s) can be processed to remove a modulated portion of the signal generated in response to light produced by the Stokes laser 906. In this manner, the processed signal can represent light produced by Raman scattering of the pump laser 904. The analyte estimation system 110 can be pressed against the skin 912 of a user such that the light is projected into the skin of a user and molecules included in the skin and/or tissue of the user can emit light into the photodiode 910.

In this example, the analyte estimation system 110 can include two or more windows 924. The light from both sources 904 and 906 can be projected with beam divergence of 45 degrees through the two or more windows 924. Other angles can be used.

FIG. 9C illustrates an example configuration of a system for detecting analytes in the user's skin in accordance with example embodiments of the present disclosure. In this example, the relevant portion of the computing system includes two printed circuit boards (PCBs). A first PCB 902 can include two light sources (VCSELs). A first light source 904 can include a pump laser that can produce light at one or more first wavelengths and a second light source 906 can include a Stokes laser that can produce light at one or more second wavelengths. The second wavelength(s) can be associated with the Raman signature of a target analyte. The analyte estimation system 110 includes two or more windows and each source passes their associated light through a respective window 932 or 934.

The second printed circuit board 908 can include one or more photodiodes 910 that are configured to detect light. The one or more photodiodes 910 can produce a signal based on the detected light. In some examples, the signal produced by the photodiode(s) can be processed to remove a modulated portion of the signal generated in response to light produced by the Stokes laser 906. In this manner, the processed signal can represent light produced by Raman scattering of the pump laser 904. The analyte estimation system 110 can be pressed against the skin 912 of a user such that the light is projected into the skin of a user and molecules included in the skin and/or tissue of the user can emit light into the photodiode 910.

FIG. 9D illustrates an example configuration of a system 940 for detecting analytes in the user's skin in accordance with example embodiments of the present disclosure. In this example, the relevant portion of the computing system includes two printed circuit boards (PCBs). A first PCB 902 can include two light sources (VCSELs). A first light source 904 can include a pump laser that can produce light at one or more first wavelengths and a second light source 906 can include a Stokes laser that can produce light at one or more second wavelengths. The second wavelength(s) can be associated with the Raman signature of a target analyte.

The second printed circuit board 908 can include one or more photodiodes 910 that are configured to detect light. The one or more photodiodes 910 can produce a signal based on the detected light. In some examples, the signal produced by the photodiode(s) can be processed to remove a modulated portion of the signal generated in response to light produced by the Stokes laser 906. In this manner, the processed signal can represent light produced by Raman scattering of the pump laser 904. The analyte estimation system 110 can be pressed against the skin 912 of a user such that the light is projected into the skin of a user and molecules included in the skin and/or tissue of the user can emit light into the photodiode 910.

The analyte estimation system 110 includes a single window 914 through which the light from both sources passes to interact with the skin 912 of a user and light emitted from the skin 912 of the user can pass through to the photodiode 910. Thus, the light is projected through the window towards a user's skin 912. Light can be emitted from the skin 912 of a user such that it passes through one or more filters to the photodiode 910. The width of the area in which the analyte estimation system 110 contacts the skin 912 is 5 millimeters.

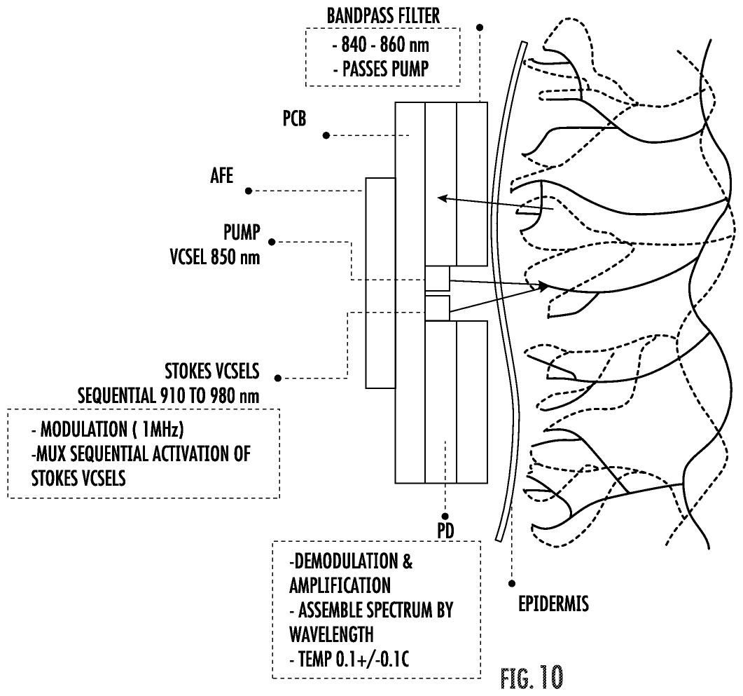

FIG. 10 illustrates an example system for detecting the presence of an analyte in the skin of the user in accordance with example embodiments of the present disclosure. The system can include a pump laser (e.g., a VCSEL) that produces light with a wavelength of 850 nanometers.

A second light source can be one or more Stokes lasers that can produce light in the range of 910 to 980 nanometers. Light from both the pump laser and the one or more Stokes lasers can be projected towards the skin of a user where it will encounter cells and blood vessels that contain a plurality of molecules. At least some of the light produced by the pump laser can excite a molecule in the skin of the users and be Raman scattered such that the photons emitted by the molecule have a different wavelength than the incoming light.

The system can include a bandpass filter that filters out light emitted from the epidermis of the user to remove light in a wavelength that is outside of the Stokes range. By filtering out wavelengths outside the Stokes range, the system can ensure that the measured light can be used to identify the Raman signature of any analytes in the skin and/or blood of the user. The nonfiltered light can then be sensed by a photodiode. The photodiode can generate an electrical signal. The electrical signal can be demodulated (e.g., using a lock-in amplifier) and amplified. In addition, if the Stokes lasers produce light at a plurality of wavelengths, the photodiode can assemble a spectrum from the various wavelengths. The amplified, demodulated, and assembled information can be analyzed to determine what molecules are present in the skin of the user and in what concentration.

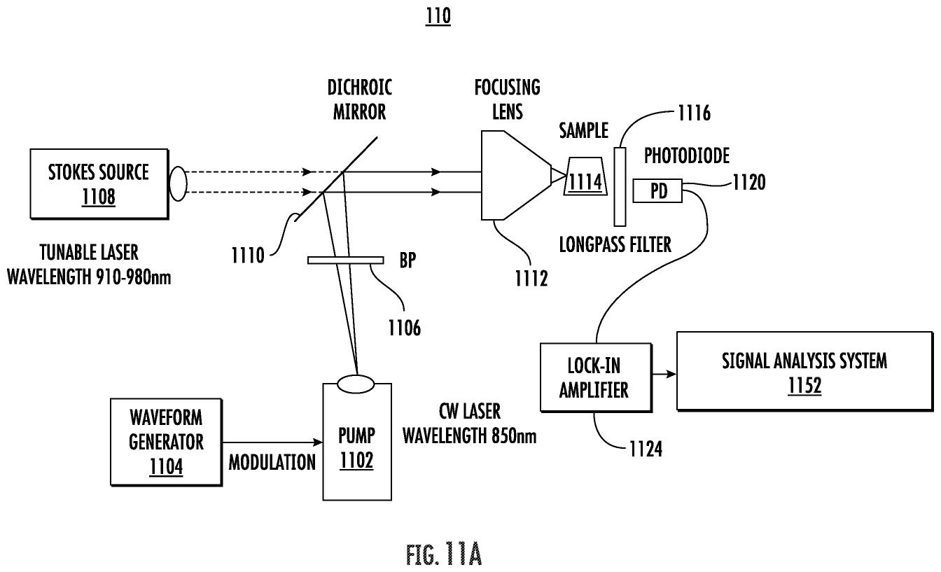

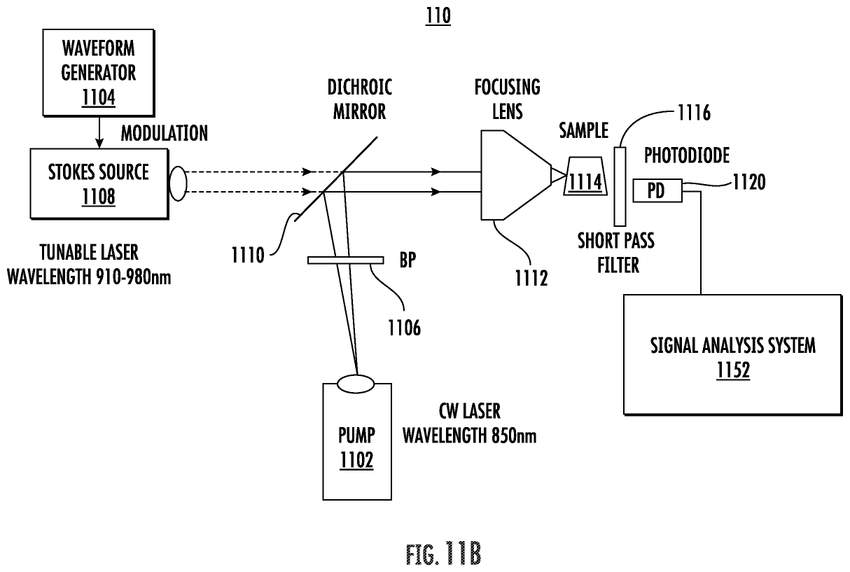

FIG. 11A represents an example analyte estimation system 110 in accordance with example embodiments of the present disclosure. In this example, the analyte estimation system 110 can include a pump laser 1102. The pump laser 1102 can be referred to as a first light source. In some examples, the pump laser 1102 can produce light with a wavelength of 850 nanometers although other wavelengths may be used. In some examples, the pump laser 1102 can take the output of a waveform generator 1104 as input. The waveform generator 1104 can produce a signal to modulate the amplitude of the light produced by the pump laser 1102. In this way, the light produced by the pump laser 1102 can be distinguished from light produced from other light sources. Thus, the analyte estimation system 110 can determine whether the wavelength of particular light is the result of Raman scattering based on the modulation.

The light produced by the pump laser 1102 can pass through a bandpass filter 1106. The bandpass filter 1106 can ensure that only light within a particular frequency (e.g., the frequency associated with 850 nanometers) passes through the filter to the sample tissue. For example, the bandpass filter can ensure that only light with a wavelength of 850 nanometers passes through the filter to the sample tissue. The analyte estimation system 110 can also include a Stokes laser 1108 (e.g., referred to as a second light source) that produces light at a narrow band around one or more second wavelengths.

In some examples, the Stokes laser 1108 can be a tunable light source. A tunable light source can be controlled to produce light at a narrow band of wavelengths around any wavelength (e.g., within 0.1 nanometers of the target wavelength) within a given predetermined range. In some examples, the tunable light source can be controlled to produce a narrow band light that sweeps through a range of wavelengths from 910 nanometers to 980 nanometers. In another example, the Stokes laser 1108 can include a plurality of different light sources (e.g., laser diodes or other light-emitting diodes), each configured to output light with a particular wavelength associated with a Raman signature of a particular analyte. For example, the Stokes lasers 1108 can include a set of VCSELs, each tuned to provide light at a different wavelength within a Stokes range of an analyte.

In some examples, the plurality of Stokes lasers 1108 can be activated one at a time such that only one Stokes laser 1108 is activated at any particular point. In other examples, a plurality of Stokes lasers 1108 can be activated simultaneously.

In this example, respective Stokes lasers 1108 at different respective light wavelengths can be amplitude-modulated at different respective time frequencies, and then the response for each different respective light wavelength can be extracted from the combined-wavelength measured signal using time-based Fourier transform or other demodulation techniques, Exemplary time modulation frequencies for amplitude-modulating the different Stokes lasers 1108 can be in the range of 10 kHz-1 MHz, although the scope of the present teachings is not so limited.

The analyte estimation system 110 can include a dichroic mirror 1110 that is configured to ensure that light emitted from the pump laser 1102 and the Stokes laser 1108 is projected in the same direction. The dichroic mirror 1110 can, for example, allow light from either the pump laser 1102 or the Stokes laser 1108 to pass through while reflecting the light from the other source. By orienting the dichroic mirror 1110 correctly, the light from both sources can be caused to be projected in the same direction.

The analyte estimation system 110 can include a focusing lens 1112 that causes the light from both the pump laser 1102 and the Stokes laser 1108 to be focused and ensure it is directed towards the target sample. Once the light from the pump laser 1102 and the Stokes laser 1108 has interacted with the sample 1114, the sample 1114 can emit light that can be filtered by one or more filters 1116. For example, the emitted light can pass through a long pass filter, which can filter out light with the first wavelength that was produced by the pump laser 1102 and allow light with the second wavelength(s) to pass through. In this way, the light from the Stokes laser 1108 and any light from the pump laser 1102 that has been Raman scattered can be passed through the filter 1116.

The analyte estimation system 110 can include a photodiode 1120 that detects light that passes through the long-pass filter. In some examples, the photodiode 1120 can be configured to measure light at any wavelength within a predetermined range of wavelengths. The light detected by the photodiode 1120 can be used to generate an electrical signal. The electrical signal can retain characteristics of the light based on which it was generated. For example, if a portion of the detect light is modulated by amplitude, the resulting electrical signal can include both a direct current portion (e.g., associated with the unmodulated light) and an alternating current (AC) portion, associated with the modulated light).

In some examples, the amount of Raman scattering can be determined by using a lock-in amplifier to distinguish the modulated portion of the electrical signal from the unmodulated portion of the electric signal. The modulated portion of the light can be determined to be associated with stimulated Raman gain (SRG) that represents the amount of light from the pump laser 1102 that was scattered (e.g., emitted from the target material at a different wavelength than the light generated by the pump laser) through Raman scattering such that the emitted light has a different wavelength than the incoming light.

The remaining electrical signal can be passed from the lock-in amplifier 1124 to the rest of the computing device for analysis. Removing the light generated by the pump laser 1102 can allow the analyte estimation system 110 to accurately calculate the stimulated Raman gain. In some examples, the stimulated Raman gain can be calculated using a signal analysis system 1152. This method can allow noninvasive measurement of a variety of different analytes including but not limited to glucose, ethanol, lipids, hemoglobin, lactate, cortisol, and so on. In other examples, a spectrograph is not used.

FIG. 11B represents an example analyte estimation system 110 in accordance with example embodiments of the present disclosure. In this example, the Stokes source 1108 is modulated, rather than the pump laser 1102. Similar to the configuration in FIG. 11A, the light from the pump laser 1102 and the modulated light from the Stokes source 1108 can pass through a dichroic mirror 1110 and a focusing lens 1112 into the sample material 1114.

In this configuration, the system includes a short pass filter 1150, which can filter out light with the second wavelength(s) (e.g. light from the Stokes source 1108) and allow light with the first wavelength from the pump laser 1102 to pass through.

The light detected by the photodiode 1120 can be used to generate an electrical signal. The electrical signal can be passed to a signal analysis system 1152 for analysis. The electrical signal is generated based on Rayleigh scattered light emitted from the target material. As noted above, the Rayleigh scattered light has the same wavelength as light generated by the pump laser 1102. As the Stokes source 1108 is modulated, the intensity of the Rayleigh scattered light can vary. In one specific example, if the amplitude of the Stokes source 1108 is at a low point (e.g., when the modulated amplitude reaches zero), the amount of Rayleigh scattered light emitted by the target material can reach a high intensity level (because less light is Raman scattered without the Stokes source 1108). Similarly, when the amplitude of the Stokes source 1108 reaches its high value during modulation, the intensity of Rayleigh scattered light emitted by the target material at the wavelength associated with the pump source 1102 may reach a low point (as more light is Raman scattered with the Stokes source 1108 being at peak intensity). The electrical signal that represents the intensity of light at the pump wavelength (because other wavelengths are filtered out by the short pass filter 1108) can be measured at the high point (e.g., when the intensity of the Rayleigh scattered light is at its maximum) and at the low point (e.g., when the intensity of the Rayleigh scattered light is at its minimum).

The difference in the electric signal between the high point and the low point can be measured by the signal analysis system 1152 to determine the amount of light that is Raman scattered. This amount can be referred to as the stimulated Raman loss (SRL). The stimulated Raman loss can represent the amount of light that, after exciting a molecule to a higher energy level in the target tissue, was emitted with a different wavelength.

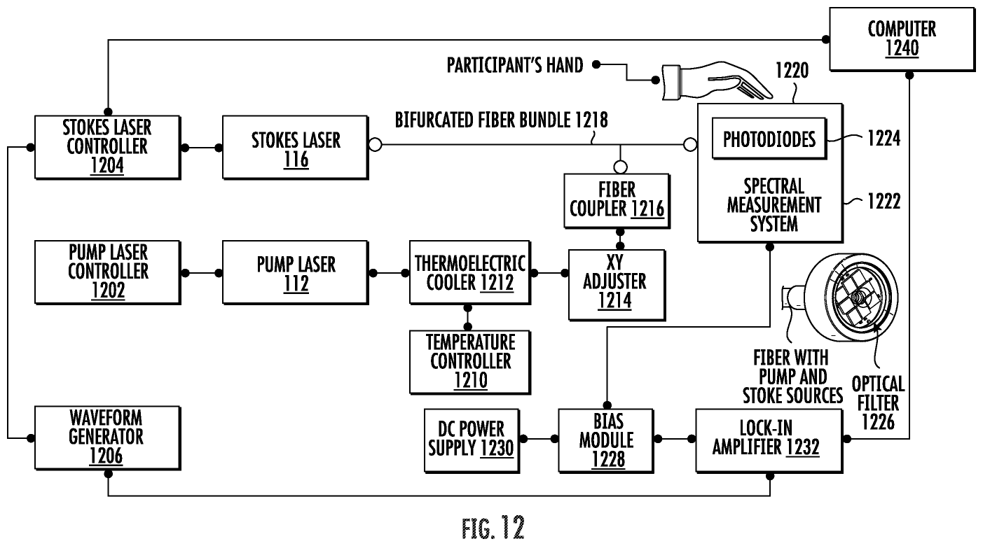

FIG. 12 illustrates an example system for non-invasively measuring analytes within a user's body in accordance with some example embodiments of the present disclosure. In accordance with example embodiments of the present disclosure. In this example, the analyte estimation system 110 can include two light sources. The first light source can be a pump laser 112 that can generate light with a first wavelength. The second light source can be one or more Stokes lasers 116. In FIG. 12, the Stokes laser 116 can be a single tunable laser that can generate a narrow band of light around any wavelength in a range of wavelengths. In another example, the Stokes lasers 116 can include a plurality of different laser diodes that each generate light at a particular wavelength and can be selectively turned on and off as needed during the detection process.

A pump laser controller 1202 can be associated with a pump laser 112. The pump laser controller 1202 can determine when the pump laser 112 is turned on and how long it remains on. A Stokes laser controller 1204 can be associated with the Stokes laser 116 and can control when the Stokes laser is 1204 turned on and, if the Stokes laser 116 is a tunable laser, what wavelength of light the Stokes laser 116 is outputting at any particular time.

A waveform generator 1206 can generate a waveform that is provided to the Stokes laser controller 1204. This waveform can be used to modulate the light produced by the Stokes laser 116. Modulating the output of the Stokes laser 116 can ensure that the light emitted by the Stokes laser 116 and the light emitted by the pump laser 112 can be separated from one another at a later point in the process.

A temperature controller 1210 can control a thermoelectric cooler 1212. The thermoelectric cooler 1212 can adjust the output of the pump laser 112 as directed by the temperature controller 1210. An XY adjuster 1214 can control the direction of the light emitted by the pump laser 112 and allow a fiber coupler 1216 to provide the light generated by the pump laser 112 into a bifurcated fiber bundle associated with the Stokes laser 116.

The light combined in the bifurcated fiber bundle 1218 can be projected towards the target sample 1220 which in this case is a participant's hand. The light can be emitted from the participant's hand towards a spectral measurement system 1222. The spectral measurement system 1222 can include one or more photodiodes 1224. In some examples, the photodiodes 1224 have an associated optical filter 1226. The optical filter 1226 can remove wavelengths of light that are not needed by the spectral measurement system 1222. The output of the spectral measurement system 1222 is provided to a bias module 1228. The biased module has a DC power supply 1230. A lock-in amplifier 1232 can remove modulated light such that the unmodulated light can be isolated and analyzed.

The output of the lock-in amplifier can be provided to a computer 1240. The computer 1240 can analyze the data to determine the presence or absence of molecules of interest in the target tissue which is, in this case, the participant's hand.

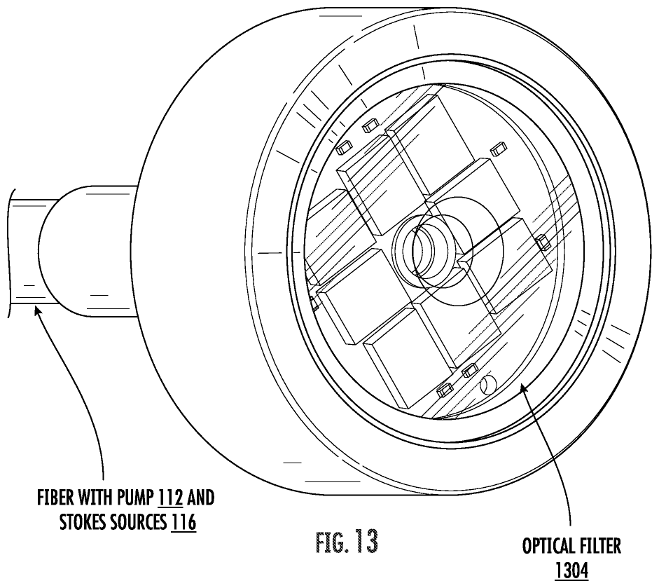

FIG. 13 illustrates an example system with light sources and photodiodes in accordance with example embodiments of the disclosure. As can be seen, the two light sources can include a pump laser 112 and a Stokes laser 116 that are positioned to project light through a fiber 1302 in a detecting unit. The light passes forward through the opening into the skin of a user. The skin of a user can emit light towards the sensor unit. The sensing unit includes an optical filter 1304 that can filter out light of certain wavelengths. The plurality of photodiodes can then detect the emitted light that has not been filtered. Information gathered by the photodiodes can be transmitted back to a computing system to be analyzed to determine the contents of the tissue into which the light was projected.

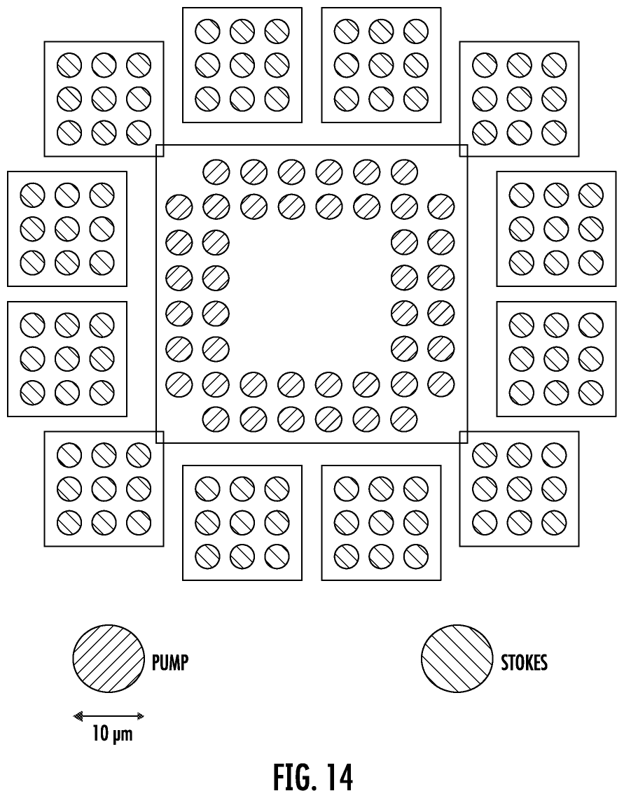

FIG. 14 illustrates a layout of a plurality of light-producing sources in accordance with some example embodiments of the present disclosure. A plurality of pump lasers can be centered in the middle providing light at a consistent wavelength. A pump laser and one or more Stokes lasers can be arranged in a pattern such that the Stokes lasers provide a plurality of different wavelengths of light. In some examples, the wavelengths of light provided by the Stokes lasers are associated with molecules of interest.

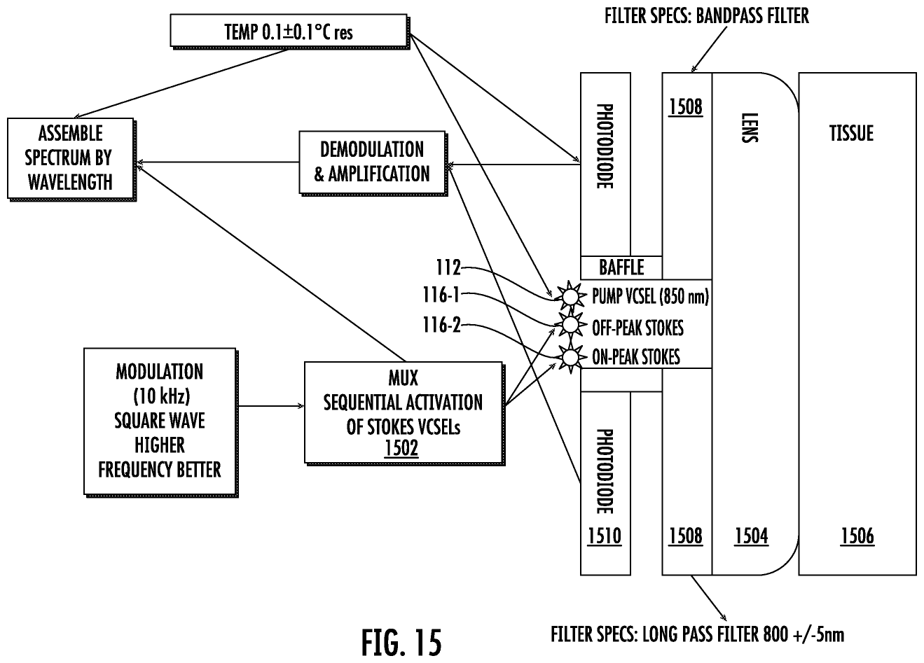

FIG. 15 illustrates an example analyte detection system 110 in accordance with some example embodiments of the present disclosure. The analyte estimation system 110 can include a pump laser 112 (e.g., a VCSEL) and two Stokes lasers 116 (e.g., an off-peak Stokes laser 116-1 and an on-peak Stokes laser 116-2). However, in other configurations, the Stokes laser 116 can include a plurality of Stokes lasers, each one configured to generate light with a particular wavelength. One or more outputs of the pump laser 112 and/or the Stokes lasers 116 may be modulated using a square wave at 10 kilohertz or higher. In general, a higher frequency is better when using modulation to distinguish light from different sources. A multiplexer (MUX) 1502 can be used to sequentially activate the two or more Stokes lasers 116.

Thus, the pump laser 112 consistently produces light at 850 millimeters and the two or more Stokes lasers 116 can be sequentially activated to produce light with a narrow band centered around specific wavelengths. The light from the pump lasers 112 and two or more Stokes lasers 116 are projected forward through a focusing lens 1504 to the tissue of a user 1506. The tissue of a user can emit light that passes through one or more filters 1508. For example, the light can pass through a bandpass filter 1508 that restricts the light that passes through to a predefined band of wavelengths or a long pass filter that filters out one or more wavelengths of light.

Once the light has passed through the filters, one or more photodiodes 1510 can detect the light (e.g., detecting the intensity of light with a particular wavelength or the number of photons with a particular wavelength). The photodiodes can generate electrical signals based on the interaction of photons with the photodiode 1510. The electrical signals can be demodulated, amplified, and transmitted to the computing device that can assemble an entire spectrum of light by wavelength. The spectrum can be analyzed to determine, based on the amounts of light at various points in the spectrum (e.g., the Raman signature), one or more analytes present in the tissue of the user. As noted above, if a plurality of laser diodes are simultaneously activated and provide light at a respective plurality of Stokes frequencies, each laser diode can be modulated at a different respective frequency. The resulting signal can be analyzed using a Fourier transform (e.g., fast Fourier transform) to determine the intensity of light at each different wavelength.

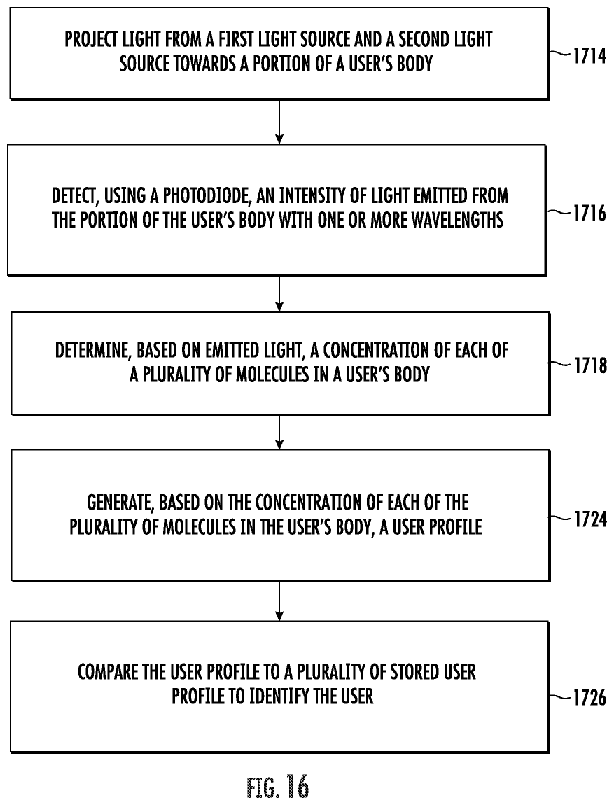

FIG. 16 is a flowchart depicting an example process of detecting molecules within a target in accordance with example embodiments of the present disclosure. One or more portion(s) of the method can be implemented by one or more computing devices such as, for example, the computing devices described herein. Moreover, one or more portion(s) of the method can be implemented as an algorithm on the hardware components of the device(s) described herein. FIG. 16 depicts elements performed in a particular order for purposes of illustration and discussion. Those of ordinary skill in the art, using the disclosures provided herein, will understand that the elements of any of the methods discussed herein can be adapted, rearranged, expanded, omitted, combined, and/or modified in various ways without deviating from the scope of the present disclosure. The method can be implemented by one or more computing devices, such as one or more of the computing devices depicted in FIGS. 1, 11-14.

The computing device can project light from a first light source and a second light source towards a portion of a user's body. The first light source can project, at 1714, light with the first wavelength, and the one or more second light sources can generate light with a second wavelength. The light sources (e.g., lasers) can project the generated light onto the skin of a user. The light can interact with molecules in the skin. The molecules in the skin can emit light to the system. In some examples, the light is Raman scattered by interacting with the molecules in the user's skin.

The computing device can detect, at 1716, using a photodiode, an intensity of emitted light with a particular wavelength. For example, the photodiode can measure the intensity of light in the second wavelength. The computing device can, at 1718, determine, based on the emitted light, a concentration of each of a plurality of molecules in a user's body.

The computing device can generate, at 1724, based on the concentration of each of the plurality of molecules in the user's body, a user profile. For example, the computing device can determine the relative concentrations of hemoglobin, glucose, lipids, and so on in a particular user's body based on information from the photodiodes. This information can be compiled into a standard user profile format.

In some examples, with the user's permission, the computing device can access locally stored user profiles or user profiles available via a computer network. The computing system can compare, at 1726, the user profile to a plurality of stored user profiles to identify the user. For example, the specific concentrations of various molecules and chemicals in the user's body can serve as a fingerprint to uniquely identify each user. However, in consideration of user privacy, no profile will be generated and no comparison be done unless the user has consented.

In some examples, once the system has determined the identity of the user based on the matching user profile, the computer system can access data associated with the user account and provide that information and services to the user.

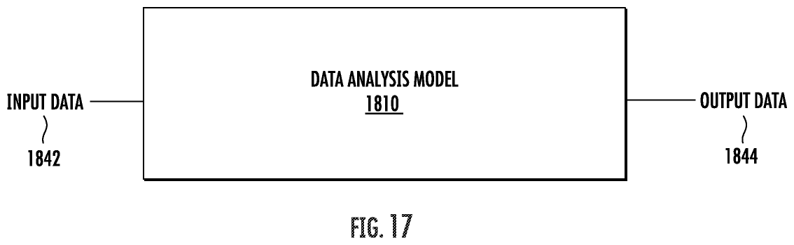

FIG. 17 depicts a block diagram of an example data analysis model 1810 according to example embodiments of the present disclosure. A machine-learned data analysis model can take information about the intensity of late at a variety of wavelengths as input 1802. For example, the data analysis model 1810 can identify the values at the expected wavelengths for glucose. Once trained, the machine-learned data analysis model can achieve a good accuracy of R2=0.84, which corresponds to approximately 30 mg/dl mean absolute error. Thus, data analysis model 1810 can output 1804 information describing whether a particular analyte is present and in what concentration.

In some examples, glucose concentrations found in human blood(<300 mg/dl, usually<140 mg/dl), result in glucose peaks that are not easily separable from the background, since the Raman signal is very weak. The model picks up the important features which align with some of the expected Raman peaks of Glucose at wavenumbers 514, 1060, 1025, 1366 cm−1, which can be clearly visible for the spectrum measured for very high Glucose concentration(>5000 mg/dl). This model can then be used to predict the glucose level for 200 new measurements with an R2 of 0.84. This corresponds to approximately 30 mg/dl mean absolute error.

In some examples, the machine-learned data analysis model 1810 can otherwise include various machine-learned models such as neural networks (e.g., deep neural networks), other types of machine-learned models, including non-linear models, and/or linear models, or binary classifiers. Neural networks can include feed-forward neural networks, recurrent neural networks (e.g., long short-term memory recurrent neural networks), convolutional neural networks, or other forms of neural networks.

A variety of training techniques can be used to train the machine-learned data analysis model 1810. Specifically, the machine-learned data analysis model 1810 can be trained using one of a plurality of semi-supervised training techniques. The machine-learned data analysis model 1810 can also be trained using a supervised training technique, such as, for example, backward propagation of errors. For example, a loss function can be backpropagated through the model(s) to update one or more parameters of the model(s) (e.g., based on a gradient of the loss function). Various loss functions can be used such as mean squared error, likelihood loss, cross entropy loss, hinge loss, and/or various other loss functions. Gradient descent techniques can be used to iteratively update the parameters over several training iterations. In some implementations, performing backward propagation of errors can include performing truncated backpropagation through time. Generalization techniques (e.g., weight decays, dropouts, etc.) can be performed to improve the generalization capability of the models being trained.

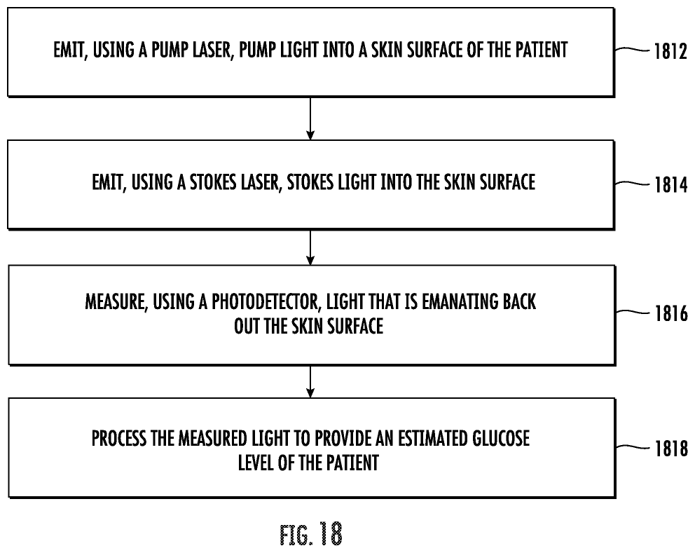

FIG. 18 is a flowchart depicting an example process of detecting analytes within a target tissue in accordance with example embodiments of the present disclosure. One or more portion(s) of the method can be implemented by one or more computing devices such as, for example, the computing devices described herein. Moreover, one or more portion(s) of the method can be implemented as an algorithm on the hardware components of the device(s) described herein. FIG. 18 depicts elements performed in a particular order for purposes of illustration and discussion. Those of ordinary skill in the art, using the disclosures provided herein, will understand that the elements of any of the methods discussed herein can be adapted, rearranged, expanded, omitted, combined, and/or modified in various ways without deviating from the scope of the present disclosure. The method can be implemented by one or more computing devices, such as one or more of the computing devices depicted in FIGS. 1, 11-14.

A computing device for non-invasively measuring glucose levels in a user using Stimulated Raman Scattering can comprise a Ramp pump laser, a Stokes laser, and a photodetector. The computing device can, at 1802, using a pump laser emit pump light into a skin surface of the user, the pump light being at a fixed wavelength. It should be noted that while the light can be directed towards the surface of the skin, the light can be focused at a subdermal area of the user such that the light is more likely to interact with the molecules in the blood of a user. Thus, when the present disclosure indicates that light is directed to or received from the surface of the skin of a user, the target of the light can be below the surface of the skin of the user. The computing device can, at 1804, using a Stokes laser, emit Stokes light into the skin surface at a plurality of Stokes wavelengths within a window of Raman measurement wavelengths.

The Stokes source can comprise a variable wavelength narrowband laser swept continuously from one end to the other end of the window of Raman measurement wavelengths during said non-invasive glucose measuring. In some examples, the Stokes source can comprise a plurality of fixed-wavelength narrow band laser sources, each having a different center wavelength lying within the window of Raman measurement wavelengths.

In some examples, the Raman pump source and the fixed-wavelength narrow band laser sources of the Stokes source are VCSELs, and wherein no optical fibers or mirrors are used anywhere in the device. The computing device can, at 1806 and using a photodetector, measure light that is emanating back out the skin surface.

In some examples, the photodetector can detect light across a range of wavelengths including both said Raman pump source wavelength and said window of Raman measurement wavelengths, and wherein time modulation of Raman pump source, time modulation of said Stokes source, and/or different time modulations of both said Raman pump source and said Stokes source are used to allow differentiation of Raman pump wavelength light from the light having the wavelengths lying within the window of Raman measurement wavelengths.

In some examples, the photodetector is a photodiode that detects light across the entire window of Raman measurement wavelengths, and wherein the plurality of fixed-wavelength narrow band laser sources are activated one-at-a-time during said non-invasive glucose measuring.

The computing device can, at 1808, process the measured light to provide an estimated glucose level of the user. In some examples, the Stokes source emits light that is narrowband relative to said window of Raman measurement wavelengths, wherein said Stokes light comprises a plurality of emissions of said narrowband light at a respective plurality of center wavelengths ranging across said window of Raman measurement wavelengths.

The technology discussed herein refers to servers, databases, software applications, and other computer-based systems, as well as actions taken and information sent to and from such systems. One of ordinary skill in the art will recognize that the inherent flexibility of computer-based systems allows for a great variety of possible configurations, combinations, and divisions of tasks and functionality between and among components. For instance, server processes discussed herein may be implemented using a single server or multiple servers working in combination. Databases and applications may be implemented on a single system or distributed across multiple systems. Distributed components may operate sequentially or in parallel.

While the present subject matter has been described in detail with respect to specific example embodiments thereof, it will be appreciated that those skilled in the art, upon attaining an understanding of the foregoing may readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

Appendix A, which forms a part of the present disclosure and specification, describes various example features of the disclosed technology. Any (or any combination of) these features may be included in the systems, methods, and other embodiments of the present disclosure. However, the present disclosure is not limited to the embodiments described in Appendix A.

1. A device for non-invasively measuring a level of an analyte in a user using Stimulated Raman Scattering, comprising:
a Raman pump source that emits pump light toward a skin surface of the user at a pump wavelength;
a Stokes source that emits Stokes light toward the skin surface at a plurality of Stokes wavelengths within a window of Raman measurement wavelengths, wherein the Stokes light comprises a plurality of narrowband emissions at a plurality of respective center wavelengths within the window of Raman measurement wavelengths;
a photodetector that measures light that emanates from the skin surface; and
a processor that processes the measured light to provide an estimated analyte level of the analyte in the user.

2. The device of claim 1, wherein the Stokes source emits Stokes light that is narrowband relative to the window of Raman measurement wavelengths.

3. The device of claim 2, wherein the Stokes source comprises a variable wavelength narrowband laser configured to provide the plurality of narrowband emissions at the plurality of the respective center wavelengths.

4. The device of claim 3, wherein the variable wavelength narrowband laser is configured to be swept continuously from one end to another end of the window of Raman measurement wavelengths during non-invasive analyte measuring.

5. The device of claim 2, wherein the Stokes source comprises a plurality of fixed-wavelength narrowband laser sources, each having a different center wavelength within the window of Raman measurement wavelengths.

6. The device of claim 5, wherein the photodetector is a photodiode that detects light across the window of Raman measurement wavelengths, and wherein the plurality of fixed-wavelength narrowband laser sources are activated one-at-a-time during non-invasive analyte measuring.

7. The device of claim 5, wherein the photodetector detects light across a range of wavelengths including both the pump wavelength and the window of Raman measurement wavelengths, and wherein at least one of modulation of the Raman pump source, time modulation of the Stokes source, or different modulations of both the Raman pump source and the Stokes source are used to allow differentiation of Raman pump wavelength light from light having wavelengths lying within the window of Raman measurement wavelengths.

8. The device of claim 7, wherein the device includes a lock-in amplifier to distinguish a portion of a signal associated with a modulated source from a portion of a signal associated with an unmodulated source.

9. The device of claim 8, wherein processing the measured light to provide an estimated analyte level of the user further comprises:
determining a stimulated Raman gain for a particular wavelength of light based on the portion of the signal associated with the modulated source.

10. The device of claim 9, wherein processing the measured light to provide an estimated analyte level of the user further comprises:
determining a stimulated Raman gain for a plurality of wavelengths; and
generating a Raman fingerprint for the skin surface of the user.

11. The device of claim 10, wherein processing the measured light to provide an estimated analyte level of the user further comprises:
generating an estimated analyte level based on the Raman fingerprint for the skin surface of the user.

12. The device of claim 11, wherein:
the modulated source is the Raman pump source; and
the unmodulated source is the Stokes source

13. The device of claim 8, wherein processing the measured light to provide an estimated analyte level of the user further comprises:
determining a stimulated Raman loss for a particular wavelength of light based on the portion of the signal associated with the unmodulated source.

14. The device of claim 11, wherein:
the modulated source is the Stokes source; and
the unmodulated source is the Raman pump source

15. The device of claim 5, wherein the Raman pump source and the fixed-wavelength narrowband laser sources of the Stokes source are VCSELs, and wherein no optical fibers or mirrors are used anywhere in the device.

16. The device of claim 1, wherein the photodetector is a broad-range photodiode.

17. The device of claim 1, wherein the analyte is glucose.

18. A computer-implemented method for non-invasively measuring a level of an analyte in a user using Stimulated Raman Scattering, comprising:
emitting, by a Raman pump source, pump light toward a skin surface of the user at a pump wavelength;
emitting, by a Stokes source, Stokes light toward the skin surface at a plurality of Stokes wavelengths within a window of Raman measurement wavelengths, wherein the Stokes light comprises a plurality of narrowband emissions at a respective plurality of center wavelengths ranging across the window of Raman measurement wavelengths;
measuring, by a photodetector, light that emanates from the skin surface; and
processing, by a processor, the measured light to provide an estimated analyte level of the user.

19. The computer-implemented method of claim 18, wherein the Stokes source emits Stokes light that is narrowband relative to the window of Raman measurement wavelengths.

20. An analyte estimation system, the system comprising:
a Raman pump source that emits pump light toward a skin surface of a user at a pump wavelength;
a Stokes source that emits Stokes light toward the skin surface at a plurality of Stokes wavelengths within a window of Raman measurement wavelengths, wherein the Stokes light comprises a plurality of narrowband emissions at a respective plurality of center wavelengths ranging across the window of Raman measurement wavelengths;
a photodetector that measures light that emanates from the skin surface; and
a processor that processes the measured light to provide an estimated analyte level of the user.
